(19) United States
(12) Patent Application Publication
(10) Pub. No.: US 2024/0252218 A1
(43) Pub. Date: Aug. 1, 2024

(54) Telescoping Screw Head with Retention Feature

(71) Applicant: Stryker European Operations Limited, Carrigtwohill (IE)

(72) Inventors: Matthias Bartsch, Schoenkirchen (DE); Tobias Boehm, Kiel[lf] (DE)

(21) Appl. No.: 18/424,280

(22) Filed: Jan. 26, 2024

Related U.S. Application Data
(60) Provisional application No. 63/441,965, filed on Jan. 30, 2023.

(51) Int. Cl. A61B 17/86; A61B 17/00; A61B 17/56

(52) U.S. Cl. CPC A61B 17/8685; A61B 17/8695; A61B 2017/00991; A61B 2017/564; A61B 2017/8655

(57) ABSTRACT

The present application relates to a bone fixation apparatus comprising a shaft portion, a head portion, and a spring. The shaft portion includes bone engaging threads. The head portion is associated with the shaft portion such that the head portion can move with respect to the shaft portion. The spring provides a force on both the shaft portion and the head portion and is enclosed between the head portion and the shaft portion.

### CROSS-REFERENCE TO RELATED APPLICATIONS

This application claims the benefit of the filing date of U.S. Provisional Patent Application No. 63/441,965 filed Jan. 30, 2023, the disclosure of which is hereby incorporated herein by reference.

### BACKGROUND OF THE INVENTION

The present disclosure relates to the usage of intramedullary nails for treatment of humeral fractures. While discussed largely in connection with use in the humerus, the present disclosure has applicability to other types of screws and nails as well.

Intermedullary nails have been established as a standard treatment for proximal humeral fractures, along with the more conservative approach of using locking plates. Such treatment has shown good clinical outcomes for both 2-part proximal humerus fractures and more complex fracture patterns. Nevertheless, high complications rates and compromised functional outcomes, e.g., impaired range of motion remain associated with such treatment. While these outcomes are commonly tolerated in the most elderly patient groups, mechanical or biologically induced complications (e.g., avascular necrosis) in other patients frequently require further surgical treatment.

One mechanical failure that can result is the unintended back-out of locking screws. While the occurrence of such failure is limited, the treatment often requires surgical intervention. As such, advancements have been made to reduce unintended back-out of locking screws. For instance, nails have been provided with threaded interfaces and even polymer rings or the like to increase the loosening torque of the screws. However, even with such advancements, unintended back-out continues to occur. Additionally, when bone resorption processes take place, the risk of unintended back-out might be increased because of in vivo reduction of screw-fragment pre-tension of the bone-nail-screw construct.

Thus, it would be desirable to provide countermeasures to further reduce the risk of unintended back-out and self-loosening of locking screws, particularly in cases of bone resorption.

### BRIEF SUMMARY OF THE INVENTION

The specific examples herein relate to a bone fixation apparatus that is spring loaded to reduce the risk of unintended back-out after implantation. The spring-loaded mechanism creates axial tension during the insertion process which results in increased friction on the bone engaging threads. The axial tension created by the spring-loaded mechanism additionally allows the bone fixation apparatus to self-adapt to maintain intimate contact with the bone tissue under changing conditions, e.g., but not limited to, during the bone resorption process.

The examples herein further refer to a bone fixation apparatus that reduces the risk of self-loosening by having a head portion and a shaft portion that are rotationally decoupled. This decoupling arrangement prevents rotational force that is applied to the head portion in vivo from transferring to the shaft portion. Additionally, the examples refer to a bone fixation apparatus with a washer capable of increasing fragment stability in circumstances of poor bone quality. The washer being configured engage with and reinforce the bone tissue around the insertion site of the shaft portion of the bone fixation apparatus.

In accordance with an aspect, a bone fixation apparatus with a spring-loaded mechanism that creates axial tension between a head portion and a shaft portion during the insertion process. This axial tension increases the friction between the bone engaging threads of the shaft portion and the bone of the patient by pulling the bone engaging threads tight against the bone tissue. In this manner, the increased thread friction created by the spring-loaded mechanism reduces the risk of the bone fixation apparatus experiencing unintended back-out after implantation.

In some other arrangements, the bone fixation apparatus rotationally decouples the head portion and the shaft portion of the bone fixation device such that no rotational force can be transferred from head portion to the shaft portion. This arrangement prevents rotational forces applied to the head portion in vivo to be transferred to the shaft portion. In this manner, the bone fixation apparatus reduces the risk of self-loosening.

In another aspect, a bone fixation apparatus may include a shaft portion with bone engaging threads and a head portion associated with the shaft portion such that the head portion may be moved with respect to the shaft portion. Additionally, a spring may provide a force on both the shaft portion and the head portion by being enclosed between the head portion and the shaft portion.

In some arrangements, the spring may be configured to create axial tension between the shaft portion and the head portion. In some arrangements, the spring may be positioned externally to the shaft portion and internally to the head portion. In some arrangements, the head portion and shaft portion may be associated such that the head portion cannot exert a rotational force on the shaft portion. In some arrangements, the head portion may be a hollow cylinder with a first hole at a first end and a second hole at a second end. In some arrangements, the first hole of the head portion may be wider than the second hole of the head portion such that the second hole defines an inner edge. The shaft portion, in some arrangements, may further include an enlarged head extending from a top end of the shaft portion such that the enlarged head is wider than the remainder of the shaft portion and the second hole of the head portion. In some arrangements, the spring may surround the shaft portion and abut the inner edge of the head portion and the enlarged head of the shaft portion. In accordance with another aspect, the shaft portion may be configured to receive a screwdriver at a top end of the shaft portion such that rotational force can be transmitted from the screwdriver shaft to the shaft portion. In some arrangements, the head portion may include a rim extending from the first end such that the rim is wider than the remainder of the head portion. In other arrangements, the spring is positioned externally to the head portion and internally to the shaft portion. In some arrangements, the head portion may be configured to receive a screwdriver shaft at a first end. In some arrangements, the shaft may further define a rectangular cavity down the center of the shaft portion.

In accordance with another arrangement, the head portion may include a lower segment that defines a rectangular rod configured to be received by the rectangular cavity of the shaft portion such that the head portion is configured to exert a rotational force on the shaft portion. In some arrangements, a first side of the spring may be affixed to a plate that is affixed inside the rectangular cavity of the shaft portion, and a second side of the spring may be affixed to the lower segment of the head portion. In some arrangements, the plate may be pressed fitted into the rectangular cavity of the shaft portion such that the plate anchors the spring inside the rectangular cavity. In another arrangement, the bone fixation apparatus may further include a washer that defines a hole through which a portion of the bone fixation apparatus can be received.

In accordance with another aspect of the invention, the bone fixation apparatus may be assembled by a process. In the process, the spring may be inserted into the interior of the head portion, and the shaft portion may be inserted into the spring and the head portion until a top end of the shaft portion abuts the spring and the spring is encased by the head portion and the shaft portion. In some arrangements, this process may further include inserting the head portion, the spring, and the shaft portion through a hole in the washer until a first end of the head portion abuts the washer.

In accordance with yet another aspect, the bone fixation apparatus may be used during a surgical procedure. In the procedure, the shaft portion may be inserted into a bone, and causing a head portion of the bone fixation apparatus to move with respect to the shaft portion such that axial tension is generated between the head portion and the shaft portion via a spring enclosed between the head portion and the shaft portion.

In some arrangements of this additional aspect, the procedure may further include engaging threads of the shaft portion with the bone. In some arrangements, the procedure may further include engaging threads of the shaft portion and rotating the shaft portion without rotating the head portion.

In accordance with another arrangement, the bone fixation apparatus may be assembled by a process. In the process, the bone fixation apparatus may be attached to a first side of a spring to a plate, and a second side of the spring may be attached to a lower segment of a head portion. Then the plate, the spring, and the lower portion of the head portion are inserted into a cavity of a shaft portion.

### BRIEF DESCRIPTION OF THE DRAWINGS

A more complete appreciation of the subject matter of the present disclosure and the various advantages thereof may be realized by reference to the following detailed description which refers to the accompanying drawings, in which:

FIG. 1 is a perspective view of a bone fixation apparatus and screwdriver in accordance with an aspect;

FIG. 2 is a perspective view of a head portion of the bone fixation apparatus of FIG. 1;

FIG. 3 is a perspective view of a shaft portion of the bone fixation apparatus of FIG. 1;

FIG. 4 is a perspective view of a washer of the bone fixation apparatus of FIG. 1;

FIG. 5 is a perspective view of a spring of the bone fixation apparatus of FIG. 1;

FIG. 6 is a cross-sectional view of the bone fixation apparatus and screwdriver of FIG. 1;

FIG. 7 is an enlarged view of the cross-sectional view of FIG. 6 with a focus on the head portion; and

FIG. 8 is an exploded view of the bone fixation apparatus of FIG. 1;

FIG. 9 is a cross-sectional view of a bone fixation apparatus in accordance with another aspect;

FIG. 10 is a perspective view of the bone fixation apparatus of FIG. 9;

FIG. 11 is an exploded view of the bone fixation apparatus of FIG. 9; and

FIG. 12 is a cross-sectional view of a bone fixation apparatus in accordance with another aspect.

### DETAILED DESCRIPTION

As used herein unless stated otherwise, the term “anterior” means toward the front part of the body, and the term “posterior” means toward the back part of the body. When referring to specific directions in the following discussion of a certain device, the terms “proximal” and “distal” are to be understood in regard to the device's orientation and position during exemplary application to human body. Thus, the term “proximal” means closer to the operator or in a direction toward the operator, and the term “distal” means more distant from the operator or in a direction away from the operator. In addition, the terms “about,” “generally,” and “substantially” are intended to mean that deviations from absolute are included within the scope of the term so modified.

Turning now to the figures, a bone fixation apparatus is shown and will be discussed below. The particular examples shown are for the internal fixation of the bone fixation apparatus. However, the various examples of the bone fixation apparatus disclosed below are not so limited to just the specific designs and uses disclosed herein.

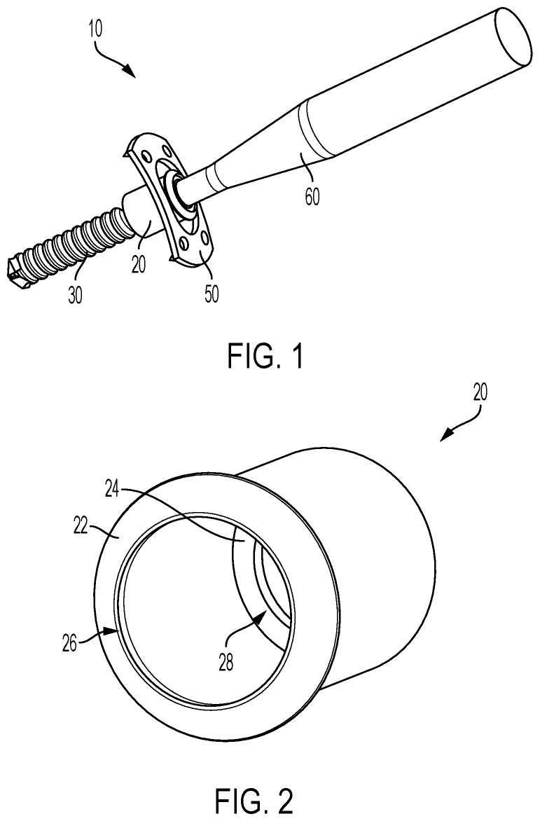

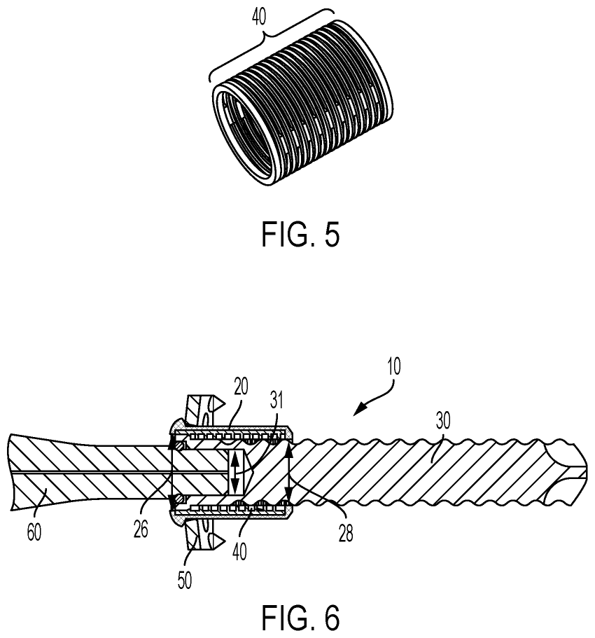

Referring now to FIG. 1, a bone fixation apparatus 10 generally includes a head portion 20, a shaft portion 30, and a spring 40 (best shown in FIG. 5) enclosed between the head portion 20 and the shaft portion 30. The spring 40 and a portion of the shaft portion 30 are inserted into the head portion 20. The shaft portion 30 is configured to receive a screwdriver 60 such that rotational force can be transferred from the screwdriver 60 to the shaft portion 30. However, the head portion 20 and the shaft portion 30 are rotationally decoupled such that no rotational force can be transmitted from the shaft portion 30 to the head portion 20 at any point before, during or after implantation. Additionally, in this example the bone fixation apparatus 10 is used with a washer 50 with a central aperture 51 large enough that most of the head portion 20 can be inserted therethrough, although not all applications will require the use of the washer 50. The bone fixation apparatus 10 can be made of any material suitable for implanting into a human body, e.g., but not limited to, titanium or titanium-alloy. An exploded view depicting each of the components of the bone fixation apparatus 10 is included in FIG. 8.

FIG. 2 focuses on the head portion 20 of the bone fixation apparatus 10. As shown, that component is a hollow cylinder with a first hole 26 at a first end and a second hole 28 at second end. The first hole 26 has a wider diameter than the second hole 28 such that the second hole 28 defines an inner edge or shoulder 24 of the head portion 20, and the first hole 26 defines an inner diameter of the head portion 20. The head portion 20 further includes a rim 22 the extends radially outward from the first end such that the rim 22 protrudes beyond the remainder of the cylindrical profile of the head portion 20.

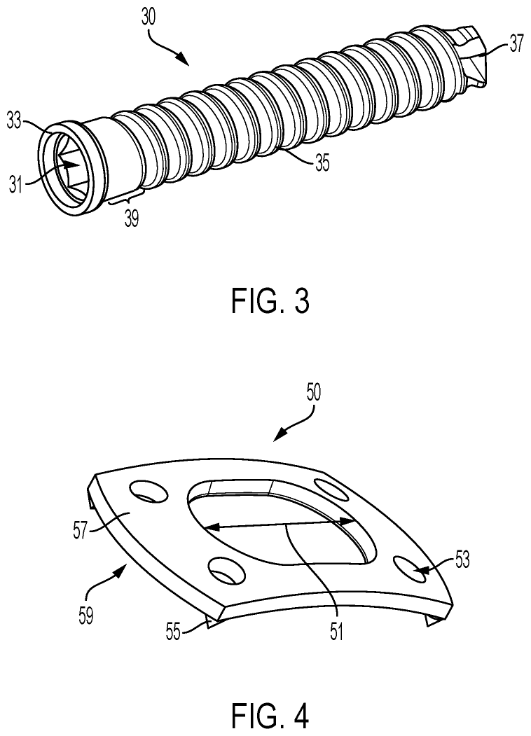

FIG. 3 focuses on the shaft portion 30 of the bone fixation apparatus 10. As shown, that component includes an elongate cylindrical rod with a top end 39 and bottom end 37. The shaft portion 30 includes bone engaging threads 35 between the top end 39 and the bottom end 37. This example of the shaft portion 30 further includes an enlarged head 33 extending from top end 39 of the shaft portion 30 such that the enlarged head 33 defines an outer diameter larger than the remainder of the shaft portion 30 and of the outer diameter of the spring 40 (discussed below). However, the outer diameter of the enlarged head 33 is less than the inner diameter of the first hole 26 of the head portion 20. Also, the shaft portion 30 defines a cavity 31 configured to receive a screwdriver such that rotational force can be transferred from the screwdriver to the shaft portion 30. Although the cavity is shown exhibiting a particular shape, it is contemplated to configure such element in any manner suitable for engaging any type of screwdriver or the like tool. Similarly, while shown as a female component it is contemplated to include a male component designed to engage a driver or other tool including a female element.

The washer 50 of the bone fixation apparatus 10 is best shown in FIG. 4 as having a rectangular shaped outer profile defining a central aperture 51 and top and bottom surfaces 57, 59. The central aperture 51 is sized and configured to receive the head portion 20, with the exception of the rim 22, which includes a larger diameter/size and radially protrudes from the head portion 20. This creates an abutting configuration when the shaft portion 30 is inserted through the washer 50. The central aperture 51 is depicted as being an oval-shaped opening with linear segments, although in other embodiments it could be circular or other shapes. The oval-shaped central aperture 51 in this example is configured to allow the washer 50 to move with respect to the head portion 20 such that the washer 50 can adjust to the bone surface underneath. The washer 50 can be configured to rotate and slide around the head portion 20 prior to complete implantation. In this manner, the washer 50 may be configured to adapt to uneven bony surfaces.

Continuing with FIG. 4, the washer 50 also includes spikes 55 that protrude toward a patient from the bottom surface of the washer 50. These structures preferably dig into the bone and provide additional stability to the construct. The washer 50 also includes secondary holes 53 that surround the central aperture 51. The secondary holes 53 can be used to receive k-wires or the like in instances where a plate is applied first and the bone fixation apparatus 10 is applied in a final stage. In other words, the secondary holes 53 can receive one or more k-wires to aid in guiding the washer 50 into place on the bone. Additionally, the washer 50 may be used to prevent the head portion 20 from penetrating the bone cortex by providing additional support. This is especially useful when the underlying bone may not be strong enough to prevent the head portion 20 from penetrating it.

FIG. 5 provides a perspective view of the spring 40 of the bone fixation apparatus 10. Spring 40 has a cylindrical profile with flat surfaces on both ends. The spring may be made of any type of metal, e.g., but not limited to, steel, copper, brass, titanium, and any alloy thereof. The length of the spring 40 should be such that the spring 40 can be encased by the head portion 30 and the shaft portion 30.

FIG. 6 provides a cross-sectional view into the interrelationship among the fully assembled head portion 20, shaft portion 30, and spring 40. The view also shows the engagement of the screwdriver 60 with the shaft portion 30. An O-ring 80 is placed within the inner diameter of the enlarged head 33 of the shaft portion 30 that is capable of receiving the shaft of the screwdriver 60. In use, the O-ring 80 allows for the bone fixation apparatus 10 to be self-retained on the screwdriver 60, with minimal force required to remove the apparatus therefrom.

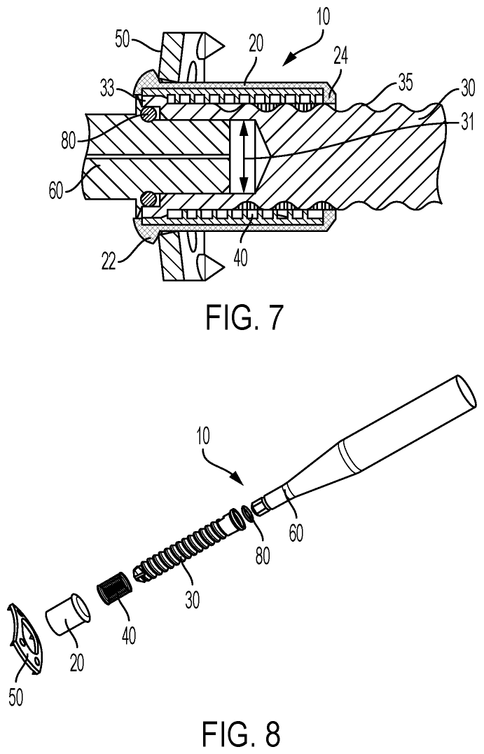

With reference to the enlarged view of FIG. 7, the bone fixation apparatus 10 is arranged such that the head portion 20 and the shaft portion 30 encase the spring 40, such that the spring 40 is sandwiched between the inner edge 24 of the head portion 20 and the enlarged head 33 of the shaft portion 30. Before implantation of the bone fixation apparatus 10, the arrangement of the spring 40 creates a pre-tension between the head portion 20 and the shaft portion 30. During the insertion process, the bone fixation apparatus 10 is lengthened as the bone engaging threads sink deeper into the bone of the patient and the head portion 20 presses against the outer surface of the bone (or the top surface of the washer 50 if the washer 50 is utilized). As the bone fixation apparatus 10 lengthens, the spring 40 is compressed between the inner edge 24 of the head portion 20 and the enlarged head 33 of the shaft portion 30, thereby gradually increasing the axial tension between the head portion 20 and the shaft portion 30. This axial tension increases the friction between the bone engaging threads 35 and the bone tissue which allows the bone engaging threads 35 to maintain close contact with the bone. This necessarily resists the loosening/back-out of the bone fixation apparatus 10.

With reference to the exploded view of FIG. 8, the bone fixation apparatus 10 is assembled by inserting the spring 40 into the head portion 20 until the spring 40 abuts the inner edge 24 of the head portion 20 (as seen in FIGS. 6-7), and subsequently inserting the shaft portion 30 through the center of the spring 40 until the enlarged head 33 of the shaft portion 30 abuts an end of the spring 40. In this example, the spring 40 is tailored to fit within the inner diameter of the head portion 20, and the shaft portion 30 may be tailored to fit within the inner diameter of the spring 40, thereby allowing the spring 40 and shaft portion 30 to fit within the head portion 20. The shaft portion 30 is also designed to receive a screwdriver 60 into the cavity 31 such that rotational force can be transferred from the screwdriver 60 to the shaft portion 30. However, the head portion 20 is rotationally decoupled to the shaft portion 30 such that rotational force cannot be transferred to the head portion 20 from the shaft portion 30.

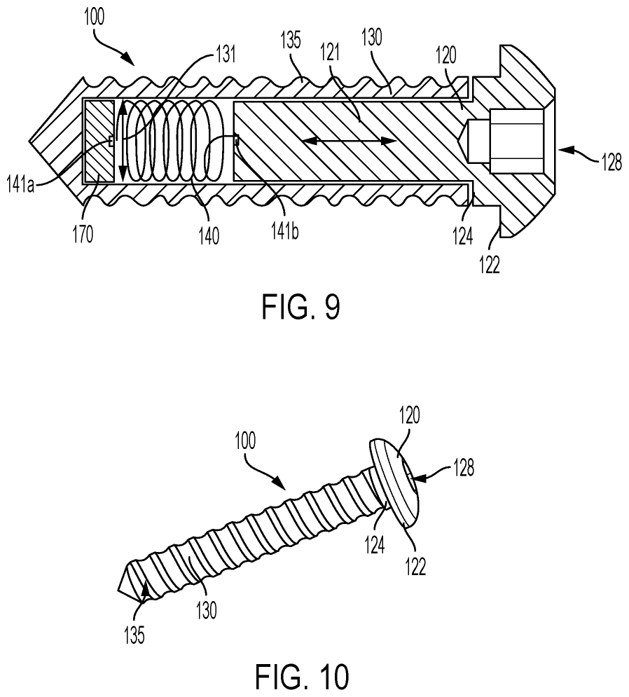

FIGS. 9-11 depict another embodiment bone fixation apparatus 100. For purposes of describing this embodiment, like reference numerals will be used when describing like elements to that of apparatus 10, but within the 100-series of numerals. For instance, the bone fixation apparatus 100 of FIG. 9 includes a head portion 120, a shaft portion 130 and spring 140. However, in this embodiment, the head portion 120 and the spring 140 are inserted into the shaft portion 130, which includes a cavity 131 configured to receive a plate 170, the spring 140, and a lower portion 121 of the head portion 120. The plate 170 may be secured to the bottom of the cavity 131 by press fit, weld, other attachment process, or can simply be free-floating within the cavity. The spring 140 may be secured to the plate and the lower portion 121 of the head portion 120, e.g., but not limited to, welding one end 141a of the spring 140 to the plate 170 and another end 141b of the spring 140 to the lower portion 121 of the head portion 120. Again, each component could also be free-floating within the cavity 131. The head portion 120 is inserted into the cavity 131 of the shaft portion 130 until the inner rim 124 of the head portion 120 abuts the shaft portion 130.

Continuing with the example of FIG. 9, the head portion 120 includes an opening 128 configured to receive the tip of a screwdriver such that rotational force can be transferred from the screwdriver to the head portion 120. The lower portion 121 of the head portion 120 is configured to engage with the walls of the cavity 131 of the shaft portion 130 such that rotational force can be transferred from head portion 120 to the shaft portion 130, thereby allowing the bone engaging threads 135 to be inserted into bone tissue as rotational force is transferred to the shaft portion 130 from a screwdriver via the head portion 120. This can be achieved by the cavity 131 of the shaft portion 130 defining a rectangular hole that corresponds with a rectangular shape formed by the lower portion 121 of the head portion 120. Additional notches or grooves with corresponding projections may be incorporated into the design of the cavity 131 of the shaft portion 130 and the lower portion 121 of the head portion 120 to facilitate transfer of rotational force from the head portion 120 to the shaft portion 130.

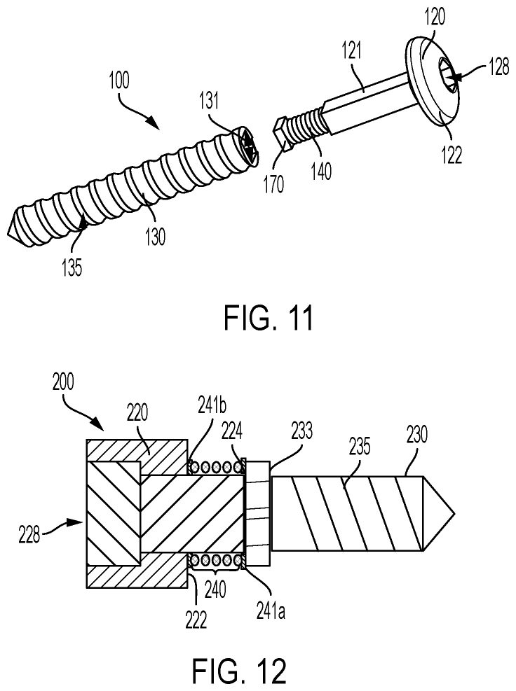

In the example of FIG. 9, during the insertion process the bone fixation apparatus 10 is lengthened and axial tension is created between the shaft portion 130 and the head portion 120, much like in the first embodiment discussed above. As the bone engaging threads 135 are inserted into bone tissue, the shaft portion 130 will continue to drive deeper into the bone tissue while the rim 122 of the head portion 120 eventually is pressed against the outer surface of the bone. At this point, the bone fixation apparatus 100 will extend as the shaft portion 130 pulls away from the head portion 120, thereby causing the axial tension between the head portion 120 and the shaft portion 130 as the spring 140 is stretched. This axial tension increases the friction between the bone engaging threads 135 and the bone tissue which allows the bone engaging threads 135 to maintain close contact with the bone. After implantation of the bone fixation apparatus, a tensile stress is built up between the head portion 20 and the shaft portion 30 such that the bone fixation apparatus 10 is shortened as bone tissue deteriorates. FIGS. 10 and 11 depict a perspective view and exploded view of the example in FIG. 9, respectively.

FIG. 12 depicts another example of a bone fixation apparatus 200. In the example of FIG. 12, the bone fixation apparatus 200 has a head portion 220, a shaft portion 230, and a spring 240. The spring 240 is sandwiched between a rim 222 of the head portion 220 and the enlarged head 233 of the shaft portion 230. However, in this example, the spring 240 is not encased by the head portion 220 and the shaft portion 230. The spring 240 is affixed to the head portion 220 and the shaft portion 230, e.g., but not limited to, welding one end 241a of the spring 240 to the enlarged head 233 of the shaft portion 230 and another end 241b of the spring 240 to the rim 222 of the head portion 220.

Continuing with FIG. 12, the head portion 220 has a rectangular lower portion that engages with a cavity of the shaft portion 230 (similar to the example in FIG. 9) such that rotational force can be transmitted from a screwdriver to the shaft portion 230 via the head portion 220. The head portion 220 being configured with receive a screwdriver in a slot 228 of the head portion 220. The bone engaging threads 235 of the shaft portion 230 engage with the bone tissue during the insertion process and drive the bone fixation apparatus 200 deeper into the bone as rotational force is transferred from the head portion 220 to the shaft portion 230. Axial tension is created when the rim 222 abuts the outer surface of the bone, which prevents further insertion of the head portion 220, while the shaft portion 230 rotates deeper into the bone.

The bone fixation apparatuses disclosed therein may come in various sizes. For instance, such may be manufactured in a variety of lengths, e.g., but not limited to, ranging between 1.5 mm to 20 mm. Moreover, it is contemplated that the diametric dimensions of the head portion and shaft portion may be manufactured in a range of sizes to meet the needs of various patients.

Additionally, the bone fixation apparatus may be made from a variety of materials. In some aspects, all the components of the bone fixation apparatus may be made from the same material such as, but not limited to, stainless steel, titanium, or titanium alloy. However, it is contemplated that head portion, shaft portion, and the other components of the bone fixation apparatus can be made of differing materials but assembled in the same apparatus. For example, the head and shaft portions may be made of titanium while the spring is made of a different metal or metal alloy to achieve a desired tension, e.g., but not limited to, brass, copper, stainless steel, titanium, or some metal alloy thereof.

The various components of the bone fixation apparatus also may be manufactured in a variety of shapes and features. For example, while the rim of the head portion of the bone fixation device is shown in the figures to be circular, it is contemplated that the head portion may take a variety of shapes, e.g., but not limited to, ovular, rectangular, triangular, or any type of polygonal shape. Similarly, the cavity of the shaft portion may be designed to conform to a specialized screwdriver tip. The distal end of the shaft portion may also include cutting edges that will allow it to cut through bone tissue during the insertion process.

Moreover, the bone fixation device may be offered in a kit of various size apparatuses, with or without associated tools and other implant devices. It is also contemplated to provide a kit that allows physicians to interchange the components of the bone fixation device intraoperatively. For example, a variety of springs, shaft portions, and washers may be combined with a certain head portion, wherein the different springs and shaft portions present different dimensions and/or features designed for a particular purpose. The kit may additionally include tools necessary to insert the bone fixation apparatus, e.g., but not limited to, screwdrivers of various sizes that match the various sizes of the components of the bone fixation apparatus in the kit.

The bone fixation device can be manufactured by various means including ways that allow for customization. The components of the bone fixation device can be manufactured with either subtractive or additive manufacturing. Additive manufacturing methods may be employed to manufacture a customized bone fixation device that is tailored specific to the condition and parameters specific to a patient. Additionally, although discussed largely in connection with use with humeral nails, it is contemplated that the devices and components disclosed herein could be used with or tailored to other uses, such as in connection with femoral nails or the like. Moreover, screws according to the present invention need not be used solely with nails, but could be implemented in connection with bone plates or other surgical screw uses.

It is to be understood that the disclosure set forth herein includes any possible combinations of the particular features set forth above, whether specifically disclosed herein or not. For example, where a particular feature is disclosed in the context of a particular aspect, arrangement, configuration, or arrangement, that feature can also be used, to the extent possible, in combination with and/or in the context of other particular aspects, arrangements, configurations, and arrangements of the technology, and in the technology generally.

Furthermore, although the technology herein has been described with reference to particular features, it is to be understood that these features are merely illustrative of the principles and applications of the present technology. It is therefore to be understood that numerous modifications, including changes in the sizes of the various features described herein, may be made to the illustrative arrangements and that other arrangements may be devised without departing from the spirit and scope of the present technology. In this regard, the present technology encompasses numerous additional features in addition to those specific features set forth in the claims below. Moreover, the foregoing disclosure should be taken by way of illustration rather than by way of limitation as the present technology is defined by the claims set forth below.

1. A bone fixation apparatus comprising:
a shaft portion including bone engaging threads,
a head portion associated with the shaft portion such that the head portion can move with respect to the shaft portion, and
a spring providing a force on both the shaft portion and the head portion, the spring being enclosed between the head portion and the shaft portion.

2. The bone fixation apparatus of claim 1, wherein the spring is configured to create axial tension between the shaft portion and the head portion.

3. The bone fixation apparatus of claim 1, wherein the spring is positioned externally to the shaft portion and internally to the head portion.

4. The bone fixation apparatus of claim 3, wherein the head portion and shaft portion are associated such that the head portion cannot exert a rotational force on the shaft portion.

5. The bone fixation apparatus of claim 3, wherein the head portion is a hollow cylinder with a first hole at a first end and a second hole at a second end.

6. The bone fixation apparatus of claim 5, wherein the first hole of the head portion is wider than the second hole of the head portion such that the second hole defines an inner edge.

7. The bone fixation apparatus of claim 6, wherein the shaft portion further includes an enlarged head extending from a top end of the shaft portion such that the enlarged head is wider than the remainder of the shaft portion and the second hole of the head portion.

8. The bone fixation apparatus of claim 7, wherein the spring surrounds the shaft portion and abuts the inner edge of the head portion and the enlarged head of the shaft portion.

9. The bone fixation member of claim 3, wherein the shaft portion is configured to receive a screwdriver shaft at a top end of the shaft portion such that rotational force can be transmitted from the screwdriver shaft to the shaft portion.

10. The bone fixation apparatus of claim 5, wherein the head portion includes a rim extending from the first end such that the rim is wider than the remainder of the head portion.

11. The bone fixation apparatus of claim 1, wherein the spring is positioned externally to the head portion and internally to the shaft portion.

12. The bone fixation apparatus of claim 11, wherein the shaft portion defines a rectangular cavity down the center of the shaft portion.

13. The bone fixation apparatus of claim 12, wherein the head portion includes a lower segment that defines a rectangular rod configured to be received by the rectangular cavity of the shaft portion such that the head portion is configured to exert a rotational force on the shaft portion.

14. The bone fixation apparatus of claim 13, wherein a first side of the spring is affixed to a plate that is affixed inside the rectangular cavity of the shaft portion, and a second side of the spring is affixed to the lower segment of the head portion.

15. The bone fixation apparatus of claim 14, wherein the plate is press fitted into the rectangular cavity of the shaft portion such that the plate anchors the spring inside the rectangular cavity.

16. The bone fixation apparatus of claim 1, further comprising a washer that defines a hole through which a portion of the bone fixation apparatus can be received.

17. A surgical method comprising,
inserting a shaft portion of a bone fixation apparatus into a bone, and
causing a head portion of a head the bone fixation apparatus to move with respect to the shaft portion,
wherein axial tension is generated between the head portion and the shaft portion via a spring enclosed between the head portion and the shaft portion.

18. The surgical method of claim 17, wherein the inserting step includes engaging threads of the shaft portion with the bone.

19. The surgical method of claim 18, wherein the inserting step includes rotating the shaft portion without rotating the head portion.

20. A method of assembling a bone fixture apparatus, the method comprising,
attaching a first side of a spring to a plate,
attaching a second side of the spring to a lower segment of a head portion, and
inserting the plate, the spring, and the lower portion of the head portion into a cavity of a shaft portion.
